(19) United States
(12) Patent Application Publication
(10) Pub. No.: US 2007/0160686 A1
(43) Pub. Date: Jul. 12, 2007

(54) Oil of coriander, oil of coriander-containing compositions having anitmicrobial and antiphlogistic effects and their use

(76) Inventors: Matthias Augustin, Hamburg (DE); Uwe Frank, St. Nikolaus (DE); Christoph Schempp, Freiburg (DE)

Correspondence Address: GREENBLUM & BERNSTEIN, P.L.C., 1950 ROLAND CLARKE PLACE, RESTON, VA, 20191, US

(21) Appl. No.: 10/588,001

(22) PCT Filed: Feb. 2, 2005
(86) PCT No.: PCT/EP05/01042

§ 371 Date: Dec. 13, 2006

(30) Foreign Application Priority Data
Feb. 2, 2004 (EP) 04002239.4

(51) Int. Cl. A61K 36/23

(52) U.S. Cl. 424/725

(57) ABSTRACT

The invention relates to the use of oil of coriander for the prophylaxis and/or therapy of inflammatory diseases and/or of microbe caused or secondary infection diseases of the skin, of the mucous membranes and of the oral cavity.

This invention relates to oil of coriander and to compositions containing oil of coriander having antimicrobial and antiphlogistic effects. Furthermore, the invention relates to the use of such compositions for prophylaxis (particularly topical) and/or therapy of inflammatory diseases, in particular of microbial diseases or of secondary infections of the skin, the mucous membranes and the oral cavity and for the simultaneous or separate prophylaxis and/or therapy of inflammations or superinfections. The invention further relates to cosmetic and medical treatment processes.

In view of the increasing development of resistant microorganisms, treatment of diseases of the skin, of the mucous membranes and of the oral cavity caused by microorganisms has become a major challenge in the human medical field. Diseases of the skin caused by microorganisms are treated with antiseptic, antibiotic, antifungal or antiviral medication. Depending on the individual situation of the person suffering from a disease, application of the medication may be topical or systemic. Microbial superinfections of the skin, as occurring in the course of atopic dermatitis or microbial eczema are treated in the same way, generally in combination with topical or systemic immunosuppressive agents (corticosteroids, immunomodulators). Diseases of the oral cavity caused by microorganisms, e.g. inflammatory gingival or periodontal diseases which, among other things, are accompanied by the generation of plaque are usually treated with antiseptic preparations such as chlorhexidine.

The disadvantages of such methods of treatment are, on the one hand, a partly high potential for the development of side effects resulting from the use of such substances and, on the other hand, the fact that where superinfections are concerned many microbes have already developed resistance against antimicrobial antiseptic agents. Moreover, there is often poor compliance among patients in taking corticosteroids (but also antibiotics/antiseptics). This may be due on the one hand, to the synthetic nature of these substances, but also to their known side effects and in some cases to their unpleasant smell, taste or the burning sensation felt on the skin, on mucous membranes and the oral cavity after application.

However patients feel differently about numerous phytotherapeutic substances, particularly about essential oils, which have found their way into everyday use as scents and flavors. Because of their natural origin and known low potential for side effects, these substances find high acceptance among patients and users.

A number of essential oils are known to have antiphlogistic, antimicrobial and/or antifungal effects. Essential oils are extracted by means of physical, preferably distillation methods from plants or their parts, for example from spice plants or their part including their fruits.

Coriander (Coriandrum sativum) belongs to the family of Apiaceae (Umbelliferae). Coriander is found in the whole Mediterranean, in Central and Eastern Europe, Eastern Asia, North and South America. The annual or bi-annual plant grows to a height of up to 20” and has thin, screw-shaped roots and light green, feathered leaves, white-pink colored blossoms and small spherical brownish seeds. In fresh condition the seeds are even and smooth and dry into small crunchy seeds. Having meridian-shaped ribs oil of coriander is extracted usually by steam distillation, from the seeds, when ripe and dry.

Coriander seeds contain 1.5% essential oils, which consist primarily of the monoterpene alcohol linalool (70%); and in addition, of α-pinene and other terpenes (borneol, campher, geraniol etc.). Other components of the seeds are oils (13 to 21%), phenol carboxylic acids, phthalides, flavonoids and coumarins.

The known antimicrobial and antifungal effects of coriander are attributed to its essential oil and flavonoid content. Recent investigations indicate a protective effect of oil of coriander against lipid peroxygenation (E. I. Stashenko et al.; Annal. Bioanal.Chem. May 2002, 373, 1-2: 70-74). Moreover, components of coriander are reported to have an inhibitory effect on the deposition of lead in bones and organs of mice by their chelate complexation effect (M. Aga et al.; J. Ethnopharmacol. Oct. 2001, 77, 2-3: 203-208).

In 1959 Maruzella demonstrated the antibacterial effect of coriander fruits against E. coli, Erwinia caravora, Mycobacterium smegmatis and Streptomyces venezuelae, as well as an antifungal effect against several fungi as, for example, Epidermophyton interdigitale, Microsporum canis and others (J. C. Maruzella, M. Freundlich; Nature 1959, 183; 972-973; J. C. Maruzella, M. Freundlich; J. Am. Pharm. Assoc. 1959, 48; 356-358).

An antibacterial effect of oil of coriander against several human-pathogenic infective microorganisms, i.e. Pseudomonas aeruginosa and E. coli, has also been demonstrated. Complete growth inhibition of the fungi Aspergillus aegypticus, Penicillium cyclopium and Trichoderma virile was observed in an agar difflusion test (S. A. Ross, N. E. ElKeltawi, S. E. Megalla; in: Fitoterapia 4: 201-205 (1998)). Vapors of oil of coriander had an antibacterial effect against Bacillus subtilis, Salmonella typhosa and particularly Mycobacterium avium.

In the public medical field, coriander is traditionally used as an anticonvulsant and as an agent against flatulence and halitosis. In addition, it is said to alleviate nervous tension and rheumatic pain.

Additionally, a study in which mice received extracts of coriander via drinking water and food demonstrated the antihyperglycemic, insulin-releasing and insulin-like activity of Coriandrum sativum (A. M. Gray, P. R. Flatt, Br. J. Nutr. Mar. 1999, 81 (3); 203-209).

Oil of coriander is well tolerated and has remarkably few side effects; contraindications or interactions with drugs are hardly known. No chromosomal aberrations were observed in Chinese hamster fibroblasts up to high concentrations of 0.125 mg/ml (M. Ishidate, T. Sofuni, K. Yoshikawa, M. Hayashi, T. Nohmi, M. Sawada, A. Matsuoka; Food Chem. Toxicol. 1984, 22 (8); 623-636).

Degenerative changes (fatty cysts) and hepatocytes with enlarged nuclei caused by unspecific stimulation of the hepatocytes due to cell degeneration (K. D. Richter, K. D. Mukherjee, N. Weber; Z. Emaihrungswiss. 1969, Sept., 35 (3); 241-248) was seen in the liver of rats receiving food rich in fat together with coriander oil. With the exception of rare contact allergies, side effects are not known after topical application, neither were any identified within the scope of the studies on the present invention.

In the fields of dermatology, dental medicine, cosmetics and mouth and dental hygiene, oil of coriander is sporadically used as an odorous and preserving agent.

The object of this invention was to find naturally occurring substances or mixtures of substances that can be used for prophylaxis and therapy of inflammatory diseases of the skin, of the mucous membranes and of the oral cavity with or without a microbial colonization/infection. A further object of this invention was to fmd naturally occurring substances or substance combinations that are suitable for simultaneous or separate prophylaxis and for therapy of inflammations or inflammatory conditions. Compositions obtained by using such substances/ substance mixtures should not have any undesired side effects or interaction with drugs; nor should they have an unpleasant smell or taste, and should, on account of the knowledge about their natural occurrence, always find high acceptance among users or patients. Surprisingly, in-house in-vitro and in-vivo research showed that oil of coriander and oil of coriander containing compositions as well as the use thereof meet all these requirements.

The invention presented here relates to oil of coriander for simultaneous or separate antiphlogistic and antimicrobial use.

This invention also relates to compositions containing oil of coriander for simultaneous or separate antiphlogistic and antimicrobial use.

This invention also relates to the use of oil of coriander for prophylaxis and/or therapy of microbial diseases of the skin, of the mucous membranes and of the oral cavity, whereby preference is given to topical application for prophylaxis and/or therapy of microbial diseases of the skin, of the mucous membranes and of the oral cavity.

The preferred embodiments of the invention are claimed in subclaims 3 to 7 and 11 to 17.

This invention also relates to the use of oil of coriander for prophylaxis and/or treatment of inflammatory diseases. Preference in particular is given to prophylaxis and/or therapy of inflammatory diseases obtainable by topical treatment.

The preferred embodiments are identified in subclaims 9 to 10 and 11 to 17.

This invention also relates to the direct use of oil of coriander for preparation of a medicament and preparation of a cosmetic composition for (particularly topical) application in prophylaxis and/or therapy of microbial diseases of the skin, of the mucous membranes and of the oral cavity.

This invention also relates to the direct use of oil of coriander for the preparation of a medication and for the preparation of a cosmetic composition for the (particularly topical) application in the prophylaxis and/or therapy of inflammatory diseases.

Furthermore, this invention relates to processes for cosmetic treatment of the skin, of the mucous membranes and of the oral cavity by administering a composition containing oil of coriander; also, to processes for prophylaxis and/or therapy of microbial diseases of the skin, of the mucous membranes and of the oral cavity by administering a composition containing oil of coriander.

Finally, the present invention also relates to processes for cosmetic treatment by administering a composition containing oil of coriander as well as to processes for the prophylaxis and/or therapy of inflammatory diseases by administering a composition containing oil of coriander. This also includes the treatment of irritated or damaged skin as well as treatment of the skin with a disturbed barrier function.

In this description and in the claims, the term “skin” refers to the organ covering the whole body.

In this description and in the claims, the term “mucous membranes” refers to the layer coating the inner part of hollow organs; this layer is kept moist by glandular secretions. In particular, the term includes the mucous membranes of the mouth and of the nose, the conjunctiva, the mucous membranes of the gastrointestinal tract and the mucous membranes of the genital area.

In this description and in the claims, the term “inflammatory diseases” refers to diseases showing acute, subacute, chronically recurrent and chronically persistent manifestations of the skin, of the mucous membranes or in the oral cavity. Clinically, inflammatory diseases are characterized by rubeosis, swelling, pain, itching and exudation, the generation of vesicles, hyperkeratosis, hypersquamation, erosions, ulcers or other substantial defects as well as scabs generation of vesicles and other rashes. Histologically, inflamed cells are found in the corium and/or in the epidermis.

In this description and in the claims the term “prophylaxis” includes all types of prevention, i.e. preventive treatment of healthy users and patients, respectively, and also treatment of users and patients who have a certain susceptibility to microbial diseases of the skin, the mucous membranes and the oral cavity, or of users and patients, with a certain liability to inflammatory diseases (persons with a so-called “pre-disposition”). Furthermore, the term “prophylaxis” also includes the preventive treatment of applicants and patients, respectively, who, on at least one occasion have suffered from a microbial disease of the skin, the mucous membranes or the oral cavity, or who have suffered from an inflammatory disease and have overcome such a disease, for example by successful treatment such as that described here and in the claims (so-called recurrent prophylaxis). Furthermore, in this description and in the claims, the term “prophylaxis” also includes cosmetic treatment as well as caring and repairing treatment of skin which is liable to irritation.

In this invention and in the claims, the term “microbial diseases of the skin, the mucous membranes and the oral cavity” includes all types of diseases of the skin, of the mucous membranes and of the oral cavity caused by microbes, for example purulent bacterial infections of the skin (pyodermia, folliculitis impetigo, erysipela) and dermatoses infected by microbes (eczema, acne, psoriasis, prurigo etc.), which enumeration, however, is not exhaustive.

In this description and the claims, the term “inflammatory diseases” or “inflammatory conditions” includes (but is not restricted to) in particular diseases and conditions such as for example different forms of eczema, (atopic eczema, seborrhea, allergic and toxic contact eczema), psoriasis and other hyperkeratotic inflammations, acute and chronic wounds, prurigo-type skin diseases, as well as rare inflammations such as, for example, Lichen ruber planus, granulomatous and parapsoriatic skin diseases, as well as the large group of autoiimmune diseases which manifest themselves in the skin. In this description and in the claims, the term “simultaneous and separate use” refers to the fact that oil of coriander and compositions containing oil of coriander have an antimicrobial effect and an antiphlogistic effect as well. Hence, an antimicrobial and also an antiphlogistic effect are achieved when using oil of coriander or compositions containing oil of coriander in accordance with the invention, in particular, when using oil of coriander or compositions containing oil of coriander in the medical field, and even more so when using oil of coriander or compositions containing oil of coriander for the preparation of medicament. Thus, either the first effect mentioned is achieved if a microbial disease of the skin, of the mucous membranes and/or of the oral cavity is present, or the second effect is achieved, if an inflammatory condition is present in the body. Both effects are achieved simultaneously or successively, and particularly in close succession, if a microbial disease of the skin, of the mucous membranes and/or of the oral cavity and an inflammatory disease is also present. Thus, the invention provides for an antiphlogistic (anti-inflammatory) effect occurring first, followed by an antimicrobial effect; however, it also provides for an antimicrobial effect occurring first followed by an antiphlogistic (anti-inflammatory) effect. It goes without saying that oil of coriander and compositions containing oil of coriander are also used in cases where the user or patient seeks only one of the two effects. In this respect, the respective other indication for use of oil of coriander or of a composition containing oil of coriander may be understood as a prophylactic treatment in the sense defined above.

In accordance with the invention, it is possible to use oil of coriander in a pure form. In this description and the claims, pure oil of coriander, refers to the oil (the substantial components of which have been mentioned above) which is recovered from coriander seeds regularly by a careful water vapor distillation procedure. In addition, presenting this invention, the term “pure oil of coriander” also includes such oils as may be obtained by admixing the pure components of natural oil of coriander, i. e. essentially in naturally relative amounts, but also in amounts which differ from the natural composition, independent of whether the single pure component(s) is/are of natural or synthetic origin. The term “pure oil of coriander” also comprises such compositions which essentially consist of oil of coriander of natural or synthetic origin, preferably in an amount >90% by weight, but preferably in an amount >95% by weight, the remainder being made up of components that do not influence the effect(s) as, for example, solvent(s) or residues thereof or decomposition products of oil of coriander or single components thereof or other components that do not influence its medical or cosmetic effect.

Pure oil of coriander may be administered directly for prophylactic or therapeutic purposes and may be administered topically in a particularly preferred embodiment. However, within the scope of this invention, it is conceivable that pure coriander oil in the farm of preparations of compositions containing oil of coriander is administered applied in combination with other suitable substances, carriers or auxiliary substances which do not have a detrimental effect on the effects of oil of coriander, particularly the antimicrobial and/or antiphlogistic effects of oil of coriander.. For example solvents (e.g. water, aqueous mixtures or solutions, e. g. physiologic saline solutions or mixtures of water and ethanol, ethanol), food products (e.g. tea, juices, mineral water), the usual bases for sprays, infusions, ointments, gels, emulsions (O/W emulsions and W/O emulsions as well) or plasters are considered suitable.

In those cases where compositions comprising oil of coriander are used, it has proved advantageous to use oil of coriander for prophylactic or therapeutic purposes in a concentration of 0.1% to 10%, preferably in a concentration of 1% to 8%, even more preferable in a concentration of 1% to 7%, for example in a concentration of 1% to 6%. The amounts indicated above are based on the total mass of the composition as administered. The composition of oil of coriander as applied or administered may comprise suitable other components, substances, carriers or auxiliary substances which do not influence the effects of oil of coriander, and in particular which do not have a detrimental effect on the antimicrobial and/or antiphlogistic properties) of oil of coriander. Suitable are, for example, solvents (e.g. water, aqueous mixtures or solutions, for example physiologic saline solution or mixtures of water and ethanol, ethanol), food products (e.g. tea, juices, mineral water, syrup), usual bases for sprays, infusions, ointments, gels, emulsions (O/W emulsions and W/O emulsions as well) or plasters. Application may take place in whatever manner desired; however in accordance with the invention, a topical application of a composition containing oil of coriander is preferred.

Application may be effected in several doses distributed over a certain period. A substantial advantage of such dosing is that oil of coriander is not toxic and is generally well tolerated. Hence, there are no limitations concerning the number of doses of compositions of oil of coriander. However, based on his skill, an expert, will know from experience which doses are administered within a certain time period in order to achieve an advantageous effect according to the invention. The compositions should preferably be administered or applied several times per day, and particularly preferred is an administration or application twice or three times per day.

In a particularly preferred embodiment one or more further antimicrobial substance(s) and/or antiphlogistic effective substance(s) are used in addition. Such compositions are advantageous in so far as two or more antimicrobial and/or antiphlogistic substances may have a complementary antimicrobial and/or antiphlogistic spectrum and/or may have an additive or even synergistic effect. In case of these combination effects, the substance ration can be altered i.e. the amount of single substances may be reduced in favor of the other substances. Moreover, by using two or more antimicrobial and/or antiphlogistic substances, it is possible, to harmonize the taste of those substances designated for use in the oral cavity, for example to avoid a too intensive a taste of coriander occurring with desired strong antimicrobial and/or antiphlogistic effect. It goes without saying that it is possible to adapt the taste through addition of further flavoring agents and odorous substances.

Oil of coriander is preferably used for prophylaxis and/or therapy of microbial diseases of the skin, particularly of diseases of the skin caused by streptococci, staphylococci (including methicillin-resistant strains of Staphylococcus aureus), corynebacteria, Pseudomonades, yeast-like fungi and molds (impetigo, wound infections, erysipelas, pyo- dermias, tinea, candidosis, acne vulgaris etc.). Oil of coriander is also assigned for a use for prophylaxis and/or therapy of diseases of the skin and/or mucous membranes caused by viruses or for complications resulting therefrom, e.g. superinfected herpes labialis. Oil of coriander may also be employed for a treatment of microbial germs (particularly bacteria) which generate unpleasant body odors, for example axillary sweat odor.

Oil of coriander is also preferably used for prophylaxis and/or therapy of microbialdiseases of the mucous membranes, particularly for diseases of the mucous membranes caused by streptococci, staphylococci (including methicillin-resistant strains of Staphylococcus aureus), Pseudomonades, E. coli, but also yeast-like fungi and molds.

In other preferred embodiments resulting from the fact that the efficacy of oil of coriander may also be extended to such microorganisms, oil of coriander is also used in the prophylaxis and/or therapy of diseases caused by commensal microorganisms of the oral cavity which are considered to be potentially human pathogenic organisms (for example for intubated A+E patients, hematologic-oncologic patients, e.g. undergoing cytostatic therapy, in cases of immune suppression, etc.). Oil of coriander is also used in the area of the oral cavity against microorganisms which contribute decisively to the development of cariogenic plaques and halitosis. Examples of such microorganisms against which oil of coriander may be used within the scope of this invention are: certain oral streptococci (S. mutans, S. mitis, S. bovis, S. agalactiae, S. constellatus, S. oralis, group G-streptococci, S. sanguis, S. intermedius, S. durans, S. salivarius, S. dysgalactiae, S. milleri, S. gordoni), Lactococcus, Stomatococcus, Actinomyces naeslundii, Actinomyces viscosus, Actinomyces israelii, Actinobacillus actinomycetemcomitans, Fusobacterium nucleatum, Porphyromonas gingivalis, Veillonella parvula as well as Capnocytophaga, Eikenella and Haemophilus species.

In a particularly preferred embodiment, oil of coriander is used for prophylaxis and/or therapy of partially microbial or superinfected diseases of the skin, as for example atopic dermatitis, microbial eczema, chronic eczema of the hands, psoriasis, prurigo, acne, etc.. Use of oil of coriander is even more preferable for prophylaxis and/or therapy of microbial diseases of the skin such as impetigo, erysipelas, pyodermia, candidiasis and tinea.

Oil of coriander is also particularly preferred for prophylaxis and/or therapy of stomatitis diseases, parodontitis diseases, dental caries plaque generation, and halitosis. Oil of coriander may also be used against certain diseases (assumedly caused by viruses), as for example aphthae and Herpes labialis.

The inhibitory effect of oil of coriander on the generation of parodontitis diseases, plaque generation resulting into dental caries and halitosis may also be detected in vivo. Therapeutic efficacy of oil of coriander against certain diseases (assumedly caused by viruses), e. g. aphthae, herpes labialis has also been observed.

In accordance with the invention, the use of oil of coriander is advantageous for a prophylaxis and/or therapy of diseases of the oral cavity, because oil of coriander inhibits the oral bacteria involved in the generation of plaque. Hence, the use of oil of coriander in the oral cavity may prevent the generation of plaque and caries.

Moreover, oil of coriander is suitable for preventive use in the eradication of multiresistant pathogens, e. g. Methicillin-resistant Staphylococcus aureus strains (MRSA strains) from the human nasopharyngeal space (or oropharyngeal space), which is an important reservoir for horizontal propagation of those pathogens in hospitals. In numerous clinical studies, it could be shown in recent years that the epidemiologic propagation of those strains in hospitals could successfully be prevented by elimination of those pathogens from the nasopharyngeal space of affected patients and/or nurses. Unfortunately, in the meantime the antimicrobial substance “mupirocin”, which is presently used and available in ointment form has lost its efficacy more and more due to the rapid development of resistance by MRSA strains. This is why new substances which might achieve said task are in high demand by clinical microbiologists, infection control specialists and epidemiologists. Surprisingly, oil of coriander proved to be extraordinarily efficient in this respect. Hence, oil of coriander is a new, highly efficient alternative to conventional mupirocin ointment.

In connection with the prevention and/or therapy of inflammatory diseases, oil of coriander and/or compositions comprising oil of coriander are successfully used in accordance with the invention for the prevention and/or therapy of eczema (atopical eczema, seborrhoic eczema, allergic or toxic contact eczema), of psoriasis and other hyperkeratotic inflammations, of acute and chronic wounds, of pruriginous diseases of the skin as well as of more rare inflammations as Lichen ruber planus, granulomatous and parapsoriatic diseases of the skin and of the large group of autoimmune diseases showing a manifestation on the skin.

In connection with the use of oil of coriander, the invention described here sets no limits to experts with respect to possible combinations of substances and their methods of administration to the patient for prophylaxis and/or therapy of microbial diseases of the skin, of the mucous membranes and of the oral cavity and/or for the prevention and/or therapy of inflammatory diseases. In accordance with the invention, use of oil of coriander in combination with the usual solvents, carrier substances, auxiliary substances, fillers, flavoring and/or fragrant substances and optionally further components is preferred. Suitable solvents were mentioned above and are, in preferred compositions of the invention, water, aqueous solutions (optionally also containing electrolytes as, for example, inorganic or organic salts like common (table) salt), organic solvents as, for example, ethanol or ethylene glycol and mixtures of organic solvents (particularly preferred: ethanol) and water in any ratio of the components, to name only a few examples. Possible carriers are, for example, unguentum leniens (W/O emulsion), DAC basic ointment, zinci pasta mollis, Eucerin cum aqua etc.. Unguentum leniens is preferred. Possible auxiliary substances are for example, emulsifiers or dispersing agents. Suitable fillers may be, for example, natural or synthetic polymers or other fillers per se known in this technical field. Possible fragrant substances are, for example, other essential oils, extracts of lavender or camomile, or menthol. In addition to the substances mentioned above, further components may be other anti-inflammatory substances and other natural or synthetic components.

In cases where oil of coriander is used for prophylaxis and/or therapy of microbial diseases of the oral cavity, due to its pleasant taste it is basically possible and preferred in accordance with the invention presented here to use the pure substance. However, one or several additional flavoring substance(s) may be added, for example peppermint oil, eucalyptus oil, curlymint oil, lemon grass oil or cinnamon oil, to name but a few.

In accordance with the invention, it is even more preferable to use oil of coriander in the form of a solution, spray, tincture, an oil-in-water emulsion or a water-in-oil emulsion. When using oil of coriander for prevention, topical prophylaxis and/or therapy of microbial diseases of the skin, it is preferable to use oil of coriander in the form of an ointment, lotion, cream or a powder. Another form of use of oil of coriander, also preferred because of the exact way in which it can be dosed, are microsomal and nanosomal preparations, i.e. preparations containing oil of coriander as such or in a form already diluted with compatible components, encapsulated in microsomes or nanosomes. Use in the form of a solution, spray, tincture, an oil-in-water emulsion, water-in-oil emulsion, ointment, lotion, cream, paste or a powder comprises use in preparations which contain further components, as carrier and auxiliary substances (known to a experts), usual agents for preservation and preservatives (known to experts for such purposes) as well as the usual effective agents, fragrant substances, coloring substances etc. known to experts for such purposes.

A particularly preferred form of the invention, is use of oil of coriander for topical prophylaxis and/or therapy of microbial diseases of the oral cavity and/or for the prophylaxis and/or therapy of inflammatory diseases, oil of coriander is employed, as an antimicrobial and, simultaneously or separately, antiphlogistic/anti-inflammatory agent in the form of a chewing mass, a chewing gum, cellulose, film, film strip, breath strip, candy, tab, pastille, tooth paste or mouth-rinsing solution. The use in a chewing mass or in a chewing gum or breath strip, respectively, is considered to be a particularly preferred embodiment. When using oil of coriander in such a way, it is particularly preferable to employ it in a concentration of 0.1% to 10%, more so in a concentration of 1% to 8%, and even more so in a concentration of 1% to 7%, for example in a concentration of 1% to 6%, each percentage being based on the overall weight of the whole composition, i.e. in the present case: of the chewing mass or chewing gum, which means an amount of 10 mg to 60 mg per gram of chewing mass, chewing gum, candy, pastille, tooth paste or mouth-rinsing solution. The invention, however, is not restricted to these concentration amounts; on the contrary, an expert may adjust concentrations of oil of coriander in such compositions in accordance with his/her skill and in accordance with further parameters (of which the presence of further components, the specific administration form, interactions with the environment, administration to certain persons are but a few examples).

In the present description and in the claims, the term “chewing mass” includes a mass which disintegrates under chewing slowly in most of the cases. Alternatively, the term “chewing mass” is understood to include, in the present description and in the claims, a mass the carrier of which does not disintegrate substantially under chewing, but only releases oil of coriander and optionally fuirther effective agents, flavoring agents, odorous agents and/or other components: In the latter case, the mass is called “chewing gum” usually. The term “candy” is meant to include, in the sense of the present description and claims, all types of candies, chocolates, sweets for sucking, lollipops etc.. The chewing masses, chewing gums, candies and pastilles may appear in different types, sizes, colors and textures.

In a preferred embodiment of the invention, the chewing mass, chewing gum, cellulosefilm, film strip, breath strip, candy, tab, pastille, tooth paste or mouth-rinsing solution may additionally contain further carrier substances, auxiliary substances, fillers, plasticizers, components, flavoring substances and/or odorous substances. Contemplated plasticizers are, for example, vegetable oil products or glycerol. All usual sweetening agents known to a skilled person and containing sugar or being free of sugar may be used as flavoring agents. In addition, essential oils may be used, for example menthol, vanillin etc.. Peppermint oil or flavor is very often used, too.

A modern chewing gum consists of corn syrup, sugar, plasticizer and flavoring agents. The basic substance is a resin, in most cases obtained from the bark of pine trees. Also, chicle is used, a type of latex from sapotill trees. Sometimes, a synthetic material may be used, too. In most of the cases, vegetable oil products or glycerol are used as the plasticizer. The most favored flavoring substance is peppermint oil. Further examples are flavors of strawberry, apple and menthol. Particularly desired nowadays are sugarfree chewing gums which have fewer calories and do not contribute to a risk of caries to the teeth. Aspartame, mannitol and sorbitol are employed as sugar substitutes. Aspartame is a highly concentrated sweetener which is obtained from aspartic acid and phenyl alanine, two natural amino acids. Mannitol and sorbitol are usually recovered from corn. Furthermore, all agents known to a skilled person to accelerate or retard the release of the components of chewing masses or chewing gums may be employed, in the same way as further components advantageous for oral, gingival or dental care, as for example grinding materials for removal of dental plaque, fluoride compounds etc..

When using such administration forms, it is particularly advantageous that the chewing mass, chewing gum, cellulose film, film strip, breath strip, candy, tab, pastille, tooth paste or mouth-rinsing solution release the oil of coriander contained therein during a more or less extended period of time which, in addition, may be adjusted and controlled by means of parameters known to an expert. This fact permits achievement of a mild and long-lasting antimicrobial and/or antiphlogistic effect in preferred embodiments of the invention. Moreover, the mild and long-lasting taste of coriander resulting from said effect is felt to be particularly pleasant by the users.

The invention, in addition, relates to the use of oil of coriander for the manufacture of a medicament for the prophylaxis and/or therapy, preferably for the topical prophylaxis and/or therapy, of inflammatory diseases of the skin, of the mucous membranes and of the oral cavity. The invention also relates to the use of oil of coriander for the manufacture of a cosmetic composition for use against inflammatory diseases and conditions for application on the skin, the mucous membranes and the introduction into the oral cavity with the aim of obtaining a cosmetic effect.

Furthermore, the invention relates to the use of oil of coriander for the manufacture of a medication for the prophylaxis and/or therapy, preferably for the topical prophylaxis and/or therapy, of inflammatory diseases. The invention also relates to the use of oil of coriander for the manufacture of a cosmetic composition for use in connection with inflammatory diseases and conditions with the aim of obtaining a cosmetic effect.

Furthermore, the invention relates to a process for the cosmetic application or incorporation of a composition containing oil of coriander onto the skin, the mucous membranes or into the oral cavity. In the course of the process, the composition containing oil of coriander is contacted to the skin, to the mucous membranes or to the inner space of the oral cavity with the aim of achieving a cosmetic effect. This may occur, for example, by taking (for example solutions, emulsions, suspensions, chewing masses, chewing gums, breath strips, cellulose strips, tabs, pastilles and candies), applying (for example solutions, ointments, creams, powders), spraying (for example solutions, powders), brushing (for example solutions), gargling (for example solutions) or any other route of application of the compositions which may be known per se to or considered suitable by a skilled person. Subsequently, the composition containing oil of coriander is allowed to exert a cosmetic effect for a time period resulting into a cosmetic effect.

Furthermore, the invention also relates to a process for the prophylaxis and/or therapy of microbial diseases of the skin, of the mucous membranes or of the oral cavity and/or for the prophylaxis and/or therapy of inflammatory diseases. In the course of such a process, a composition containing oil of coriander is administered with the aim of contacting oil of coriander and/or its derivatives and/or its metabolites to the point of the disease, preferably of the microbial disease and/or of the inflammatory disease. This may occur, for example, by taking (for example solutions, emulsions, suspensions, chewing masses, chewing gums, candies), applying (for example solutions, ointments, creams, powders), spraying (for example solutions, powders), brushing (for example solutions), gargling (for example solutions) or any other route of application of the compositions which routes may be known per se to or considered suitable by a skilled person. Subsequently, the composition containing oil of coriander and/or its derivatives and/or its metabolites is/are allowed to exert an effect for a time period assuring a prophylaxis and/or therapy of microbial diseases. Preferably, a composition containing oil of coriander is topically applied onto the skin or the mucous membranes or into the oral cavity.

In the following, the invention is firther explained by examples which examples should be understood not to limit the invention but to exemplarily explain it.

### EXAMPLES

When screening substances of vegetable origin seeming to be suitable due to antiphlogistic or antimicrobial properties, for example for an application against dermatologic indications, oil of coriander was tested together with other vegetable substances. The screening comprised
1. Systemic analysis of the literature;
2. The development of galenically suitable compositions;
3. Tolerance tests with healthy probands;
4. In-vitro tests for antimicrobial effects;
5. In-vitro tests for antiphlogistic properties;
6. First clinical observations in the form of healing attempts;
7. Experimental tests on humans for antimicrobial and/or antiphlogistic effects.

The compositions of oil of coriander proved to be effective and suitable for application against diseases of the skin in all the tests, as show by the subsequent working examples.

(1) In-vitro Studies

In several in-vitro studies, oil of coriander proved to show strong inhibitory effects against different strains of Staphylococcus aureus, Enterococcus faecalis, E. coli, Pseudomonas aeruginosa and Candida albicans (Table 1). Oil of coriander also showed efficacy against MRSA (Methicillin-resistant staphylococci). In further studies, oil of coriander showed a good efficacy against plaque-generating bacteria in the oral cavity (inter alia Streptococcus mutans, other oral streptococci).

TABLE 1
Screening result of oil of coriander compared to values derived from
the literature

| | | E. | | | C. |
|---|---|---|---|---|---|
| | S. aureus | faecalis | E. coli | P. aeruginosa | albicans |
| Screening | +++ | +++ | + | ++ | +++++ |
| (Frank, Maier, | | | | | |
| Daschner et | | | | | |
| al., internal | | | | | |
| paper 2002) | | | | | |

0 mm no inhibition
1-2 mm inhibition (+)
3-5 mm inhibition +
6-8 mm inhibition ++
9-10 mm inhibition +++

(2) Tests on Healing Inflammatory and Microbial Diseases of the Skin

In healing tests, the following compositions containing oil of coriander were used on (altogether) 35 patients up to now:
1. Oil of coriander 6.0 g inunguentum leniens ad 100.0;
2. Oil of coriander 1.0 g in unguentum leniens ad 100.0;
3. Oil of coriander 1.0 g in pasta zinci mollis;
4. Oil of coriander 1.0 g in DAB adhesive paste (DAB=German Pharmacopoeia); ad 100,0
5. Oil of coriander 1.0 g in 5% alcohol solution ad 100.0.

In these healing experiments, favorable developments and good response rates were observed for chronic inflammatory dermatoses (composition 1) in adult patients, in particular for impetigo-affected eczema; for inguinal candidosis (composition 3) as well as for children suffering from eczema (composition 2) and from bacterial superinfections (compositions 2+3). Improvements were observed for patients suffering from stomatitis, moniliasis and aphthae (compositions 4+5).

(2) Antiphlogistic Efficacy in a UV Erythema Test

A 1% oily composition of oil of coriander in unguentum leniens reduced UV-induced erythema on the healthy skin of n=40 probands, compared to the plain ointment base and to the untreated control area. The efficacy was in the range of 1% hydrocortisone (all amounts in % by weight).

(3) Clinical Screening Study

Compositions of 1% or 6% of oil of coriander in unguentum leniens showed improved efficacy against atopical eczema and nummular eczema, compared to the ointment base.

A composition of 1% of oil of coriander in pasta zinci mollis proved to be efficient against tinea diseases as well as Candida intertrigo. 1% of oil of coriander in an adhesive paste was successfully used against candida stomatitis and aphthae in an uncontrolled application.

As the above working examples show, compositions containing oil of coriander proved to have efficacy in vitro and clinically as well in the sense of having antimicrobial and antiphlogistic properties. Hence, they are extraordinarily well suited for topical prophylaxis and/or therapy of inflammatory diseases or microbial diseases or secondary infection skin diseases of the mucous membranes and of the oral cavity.

1-23. (canceled)

24. A method of treating or preventing a microbe-caused disease of at least one of skin, a mucous membrane and an oral cavity, wherein the method comprises an administration to a subject of an amount of oil of coriander which is effective for treating or preventing the microbe-caused disease.

25. The method of claim 24, wherein the microbe-caused disease is caused by at least one of gram-positive bacteria, gram-negative bacteria, yeast-like fungi and viruses.

26. The method of claim 24, wherein the microbe-caused disease comprises at least one of a bacterial infection of the skin, a fungal infection of the skin, a microbe-infected dermatose, a microbe-caused disease of a mucous membrane, a microbe-caused disease of an oral cavity and a viral disease of a mucous membrane.

27. The method of claim 24, wherein the microbe-caused disease is associated with at least one of MRSA and a multi-resistant pathogen.

28. The method of claim 24, wherein the oil of coriander or a composition comprising the oil of coriander is administered to contact a site of the microbial disease with at least one of the oil of coriander, a derivative thereof and a metabolite thereof and is allowed to exert an effect for a period of time which is sufficient for effecting a therapy of the microbial disease.

29. A method of treating or preventing an inflammatory disease, wherein the method comprises an administration to a subject of an amount of oil of coriander which is effective for treating or preventing the inflammatory disease.

30. The method of claim 29, wherein the administration comprises a topical administration.

31. The method of claim 29, wherein the inflammatory disease comprises at least one of an acute, subacute, chronically recurrent and chronically persistent condition of at least one of skin, a mucous membrane and an oral cavity.

32. The method of claim 29, wherein the inflammatory disease comprises at least one of an eczema, psoriasis, a hyperkeratosis with inflammations, an acute or chronic wound infection, Lichen ruber planus, a granulomatous and parapsoriatic disease of skin and an autoimmune disease showing manifestations on skin.

33. The method of claim 29, wherein the oil of coriander or a composition comprising the oil of coriander is administered to contact a site of the inflammatory disease with at least one of the oil of coriander, a derivative thereof and a metabolite thereof and is allowed to exert an effect for a period of time which is sufficient for effecting a therapy of the inflammatory disease.

34. A composition which is suitable for use in the method of claim 24, wherein the composition comprises oil of coriander.

35. The composition of claim 34, wherein the composition is present as a solution, a spray, a tincture, an oil-in-water emulsion or a water-in-oil emulsion.

36. The composition of claim 34, wherein the composition is present as an ointment, a lotion, a cream, a paste or a powder.

37. The composition of claim 34, wherein the composition is present as a chewing mass, a chewing gum, a cellulose film, a film strip, a breath strip, a candy, a tab, a pastille, a tooth-paste or a mouth-rinsing solution.

38. The composition of claim 34, wherein the composition comprises from 0.1% to 10% by weight of oil of coriander, based on a total weight of the composition.

39. The composition of claim 37, wherein the composition comprises from 0.1% to 10% by weight of oil of coriander, based on a total weight of the composition.

40. The composition of claim 39, wherein the composition further comprises at least one additional substance which exhibits at least one of an anti-inflammatory and an antimicrobial effect.

41. The composition of claim 34, wherein the composition comprises a cosmetic composition which is suitable for application to skin, a mucous membrane or in an oral cavity.

42. A composition which is suitable for use in the method of claim 29, wherein the composition comprises oil of coriander.

43. The composition of claim 42, wherein the composition is present as a solution, a spray, a tincture, an oil-in-water emulsion or a water-in-oil emulsion.

44. The composition of claim 42, wherein the composition is present as an ointment, a lotion, a cream, a paste or a powder.

45. The composition of claim 42, wherein the composition is present as a chewing mass, a chewing gum, a cellulose film, a film strip, a breath strip, a candy, a tab, a pastille, a tooth-paste or a mouth-rinsing solution.

46. The composition of claim 42, wherein the composition comprises from 0.1% to 10% by weight of oil of coriander, based on a total weight of the composition.

47. The composition of claim 46, wherein the composition further comprises at least one additional substance which exhibits at least one of an anti-inflammatory and an antimicrobial effect.
